(19) United States
(12) Patent Application Publication
(10) Pub. No.: US 2011/0123400 A1
(43) Pub. Date: May 26, 2011

(54) APPARATUS FOR DETECTING METAL CONCENTRATION IN AN ATMOSPHERE

(76) Inventors: Hyun-Kee Hong, Daejeon (KR); Jae-Seok Lee, Gyeonggi-do (KR); Yang-Koo Lee, Gyeonggi-do (KR); Hun-Jung Yi, Gyeonggi-do (KR); Jung-Dae Park, Gyeonggi-do (KR); Sun-Hee Park, Gyeonggi-do (KR)

(21) Appl. No.: 13/017,440

(22) Filed: Jan. 31, 2011

Related U.S. Application Data
(62) Division of application No. 12/029,612, filed on Feb. 12, 2008.

(30) Foreign Application Priority Data
Feb. 12, 2007 (KR) 10-2007-0014462

(51) Int. Cl. G01N 33/20

(52) U.S. Cl. 422/69

(57) ABSTRACT

The present invention provides an apparatus for detecting metal concentration from an area including compounding a solution that includes a metal dissolved by a solvent, and a reagent combined with metal ions dissolved in the solution and referring a difference of absorption rates between a compound of the solvent and reagent and a compound of the solution and reagent.

### CROSS-REFERENCE TO RELATED APPLICATIONS

This application is a divisional application of U.S. application Ser. No. 12/029,612, filed on Feb. 12, 2008 which claims priority to Korean Patent Application No. 10-2007-0014462, filed on Feb. 12, 2007, the disclosures of which are incorporated herein by reference in their entireties.

### FIELD OF THE INVENTION

The present invention relates to detection of metal concentration. More particularly, the present invention relates to apparatus and methods for detecting the concentration of a metal in an atmosphere.

### BACKGROUND

With the microscopic shrinking of semiconductor device patterns in recent years, it is highly desirable to maintain the lowest pollution degree in a clean room accommodating a plurality of semiconductor processing equipment. Metallic pollutants, such as copper, included in the clean room may seriously affect degradation of semiconductor device products. Such metallic pollutants in the clean room may be generated while depositing metal films on semiconductor wafers.

Although there are several ways for measuring the concentration of nonmetallic pollutants in the clean room, methods for detecting metallic pollutants are limited.

### SUMMARY OF THE INVENTION

The present invention is directed to apparatus and methods for detecting the concentration of metallic pollutants contained in an atmosphere, such as the space of a clean room.

Embodiments of the present invention include methods for detecting concentration of a metal in a region including subjecting air from the region to a solvent to allow dissolution of the metal in the solvent; irradiating light on a liquid compound of a solution, in which the metal is dissolved, combined with a reagent that is chemically combined with the metal; and detecting the concentration of the metal by measuring an absorption rate of the liquid compound. In a particular embodiment, the metal concentration is detected by: supplying air from the space to a solvent and dissolving the metal in the solvent; irradiating light on a liquid compound of a solution, in which the metal is dissolved, and a reagent that is chemically combined with the metal; and referring to an absorption rate of the liquid compound.

Embodiments of the present invention also provide methods for detecting concentration of an atmospheric metal, including combining a solution, in which the atmospheric metal is dissolved in a solvent, with a reagent that is chemically combined with metal ions contained in the solution; detecting the concentration of the metal by comparing a difference between an absorption rate of a liquid compound of the solvent and the reagent, and an absorption rate of a liquid compound of the reagent and the solution. Particularly, detecting the concentration of an atmospheric metal may include compounding a solution, in which the atmospheric metal is dissolved by a solvent, with a reagent that is chemically combined with metal ions contained in the solution; detecting the concentration of the metal with reference to a difference between an absorption rate of a liquid compound of the solvent and the reagent, and an absorption rate of a liquid compound of the reagent and the solution.

Embodiments of the present invention further include an apparatus for detecting a concentration of a metal in a region, including: a first reservoir in which the metal in the region is dissolved by a solvent; a second reservoir capable of containing a reagent chemically combined with the metal; a first unit capable of receiving a solution, in which the metal is dissolved, from the first reservoir and a reagent contained in the second reservoir, and combining the solution and the reagent; and a second unit capable of irradiating light on a liquid compound provided by the first unit and further capable of detecting the concentration of the metal by measuring an absorption rate of the liquid compound. For example, an apparatus for detecting concentration of a metal in a space may include a gas solution reservoir in which the metal of the space is dissolved by a solvent; a reagent reservoir containing a reagent chemically combined with the metal; a compounding unit receiving a solution, in which the metal is dissolved, from the gas solution reservoir and the reagent from the reagent reservoir and compounding the solution and the reagent; and a measuring unit irradiating light on a liquid compound made by the compounding unit and detecting the concentration of the metal by measuring an absorption rate of the liquid compound.

A further understanding of the nature and advantages of the present invention herein may be realized by reference to the remaining portions of the specification and the attached drawings.

### BRIEF DESCRIPTION OF THE FIGURES

Non-limiting and non-exhaustive embodiments of the present invention will be described with reference to the following figures, wherein like reference numerals refer to like parts throughout the various figures unless otherwise specified.

FIG. 1 is a schematic diagram illustrating a structure of a metal concentration detection apparatus according to embodiments of the present invention.

FIG. 2 is a schematic diagram illustrating a structure of the measuring unit shown in FIG. 1.

FIG. 3 is a graphic diagram comparatively showing an absorption rate with a compound of copperless deionized water and a chelate, and an absorption rate with a compound of copper-containing deionized water and a chelate;

FIG. 4 is a graphic diagram showing a variation of absorption rate with a compound of copper-containing deionized water and a chelate versus concentration of a copper; and

FIG. 5 is a flow chart showing a sequence of steps for detecting metal concentration according to embodiments of the present invention.

### DETAILED DESCRIPTION

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that when an element or layer is referred to as being “on”, “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numbers refer to like elements throughout. As used herein, the term “and/or” includes any and all combinations of one or more of the associated listed items.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. All publications, patent applications, patents, and other references mentioned herein are incorporated by reference in their entirety.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Moreover, it will be understood that steps comprising the methods provided herein can be performed independently or at least two steps can be combined. Additionally, steps comprising the methods provided herein, when performed independently or combined, can be performed at the same temperature and/or atmospheric pressure or at different temperatures and/or atmospheric pressures without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

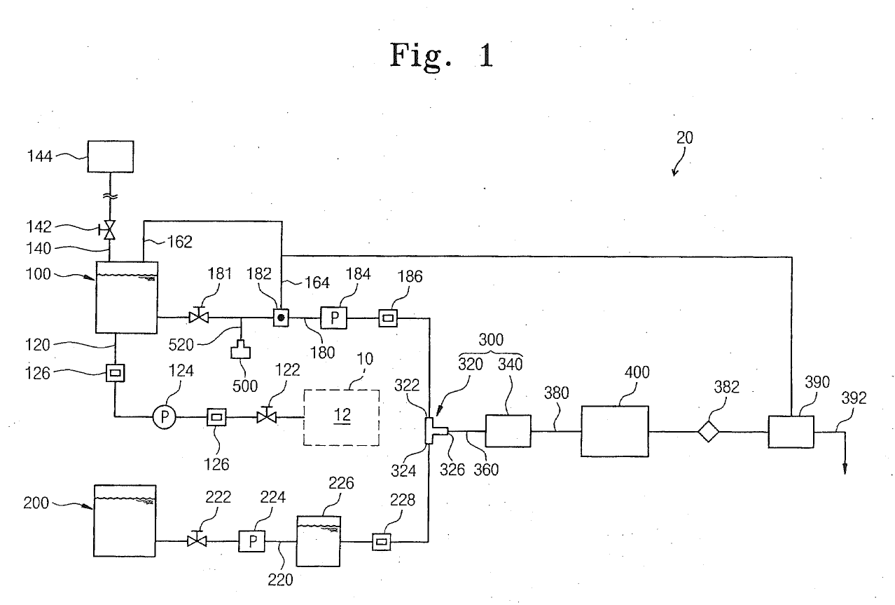

FIG. 1 is a schematic diagram illustrating a structure of a metal concentration detection apparatus 20 according to embodiments of the present invention. Referring to FIG. 1, the metal concentration detection apparatus 20 includes a gas solution reservoir 100, a reagent reservoir 200, a compounding unit 300, and a measuring unit 400. The gas solution reservoir 100 generates a solution by dissolving a predetermined quantity of air, which is abstracted from a space (hereinafter, a detection space 12) from which metal concentration is detected, in a solvent. The gas solution reservoir 100 may be associated with a mixer (not shown) for assisting an atmospheric metal to be dissolved in a solvent. The gas solution reservoir 100 may be connected to a gas inflow pipe 120. The gas inflow pipe 120 can be inserted into the gas solution reservoir 100 through the bottom wall. The gas inflow pipe 120 may be provided as a passage supplying air from the detection space 12 to the gas solution reservoir 100.

In some embodiments, the detection space 12 may be a clean room 10 in which a semiconductor fabrication process is carried out. In the clean room 10, pluralities of semiconductor fabrication equipment may be employed to conduct processing steps. An end of the gas inflow pipe 120 may be provided to a region where semiconductor fabrication equipment using a metal to be detected is located. For example, a metal to be detected may be copper, the gas inflow pipe 120 may be provided to a region where semiconductor fabrication equipment is located for depositing a copper film on a semiconductor substrate.

A pump 124 and a filter 126 may be installed in the gas inflow pipe 120. The pump 124 may provide flux pressure to the gas solution reservoir 100 so as to force air to flow into the gas solution reservoir 100. The filter 126 may remove unwanted particles from gas flowing into the gas solution reservoir 100. For example, the filter 126 may include a filter capable of removing particles from air that are larger than a predetermined size.

In the gas solution reservoir, a solvent capable of dissolving metal ions to be detected from the inflow air may be included to a predetermined level. Atmospheric metal ions may be dissolved in the solvent and the air without metal remaining at at least an upper portion of the gas solution reservoir 100. A vent 162 may be provided at the top of the gas solution reservoir 100. The air remaining at the upper portion may be exhausted from the gas solution reservoir 100 through the vent 162.

The gas solution reservoir 100 may be connected to a solution supply pipe 180 equipped with an opening valve 181. The solution supply pipe 180 may be provided to supply a solution, in which the metal ions are dissolved by the solvent, to the compounding unit 300 from the gas solution reservoir 100. A pump 184 and a filter 186 may be installed at the solution supply pipe 180. The pump 184 may provide flux pressure to force the solution of a predetermined quantity to flow into the compounding unit 300 from the gas solution reservoir 100. The pump 184 may include a piston pump. The filter 186 may function to remove unwanted substances from the solution. For example, the filter 186 may be a filter for removing foreign substances, which are larger than a predetermined size, from the solution with the metal. In addition, a bubble eliminator 182 may be installed at the solution supply pipe 186, thereby removing bubbles from the solution. Bubbles excluded from the solution by the bubble eliminator 182 may be discharged through the vent 164. The bubble eliminator 182, the pump 184, and the filter 186 may be disposed in sequence along a distance from the gas solution reservoir 100.

The solution supply pipe 180 may be connected to a sampling pipe 520. The sampling pipe 520 allows the solution to partly flow into a sampling reservoir 500 through the solution supply pipe 180. The sampling pipe 520 may be directed from the solution supply pipe 180 between the gas solution reservoir 100 and the bubble eliminator 182. The sampling reservoir 500 may store at least some of the solution for reexamining metal concentration of the solution at a later period of time.

According to embodiments of the present invention, as the solution is drained from the gas solution reservoir 100, a level of the solvent is gradually lowered in the gas solution reservoir 100. The top of the gas solution reservoir 100 is connected with a solvent supply pipe 140. The solvent supply pipe 140 includes a valve 142 for opening and closing the internal path. If a level of the solvent in the gas solution reservoir 100 becomes low enough, the solvent is supplemented to the gas solution reservoir 100 from a solvent reservoir 144 by way of the solvent supply pipe 140. Supplementation of the solvent is accomplished by installing a sensor (not shown) in the gas solution reservoir 100 to detect a level of the solvent and controlling the operation of the valve 142 by means of a controller (not shown) responding to a signal output from the sensor. The sensor is also able to selectively supplement the solvent by controlling the valve 142 by an operator who determines the time of supplementation.

The reagent reservoir 200 may store a reagent for chelating a metal to be detected. The reagent reservoir 200 may be connected to a reagent supply pipe 220 including a valve 222. The reagent supply pipe 220 may provide the reagent to the compounding unit 300 from the reagent reservoir 200. The reagent supply pipe 220 may be constructed to include a pump 224 and a filter 228. The pump 224 is capable of setting flux pressure to force a predetermined quantity of reagent to flow into the compounding unit 300. The pump 224 may include a type of piston pump. The filter 228 may operate to remove foreign substances from the reagent. For example, the filter 224 may be a filter that blocks foreign substances, which are larger than a predetermined size, from the reagent. Additionally, for the purpose of assuring a stable supply of the reagent to the compounding unit 300, the reagent supply pipe 220 may be associated with a buffering reservoir 226 for temporarily holding the reagent. The pump 224, the buffering reservoir 226, and the filter 228 may be sequentially disposed along a distance from the reagent reservoir 200.

The compounding unit 300 may be supplied with a solution generated from the gas solution reservoir 100 and with the reagent from the reagent reservoir 200 and forms a liquid compound from the solution and the reagent. According to this embodiment, the compounding unit 300 includes a first mixer 320 and a second mixer 340. The first and second mixers 320 and 340 may be joined by a link 360. The first mixer 320 may be shaped in a ‘T’ format, having two input ports 322 and 324 and a single output port 326. One of the input ports, 322, may be connected to the solution supply pipe 180 and the other of the input ports, 324, may be connected to the reagent supply pipe 220. The output port 326 may be connected to the link 360. The input ports 322 and 324 may be disposed opposite to each other on a line and the output port 326 may be disposed vertically to the line at the center between the input ports 322 and 324. With this structure, the solution and the reagent may flow into the first mixer 320, being opposite each other and further colliding. After collision, the liquid compound may be discharged through the output port 326. The second mixer 340 may be shaped as a tube. The liquid compound input through the link 360 may be remixed in the second mixer 340.

The second mixer 340 may be connected to a compound supply pipe 380. The liquid compound formed by the second mixer 340 may flow through the compound supply pipe 380. Alternatively, the compounding unit 300 may include only one of the first and second mixers 320 and 340. Further, the compounding unit 300 may include another type of mixer that is different in structure from the first mixer 320 or the second mixer 340.

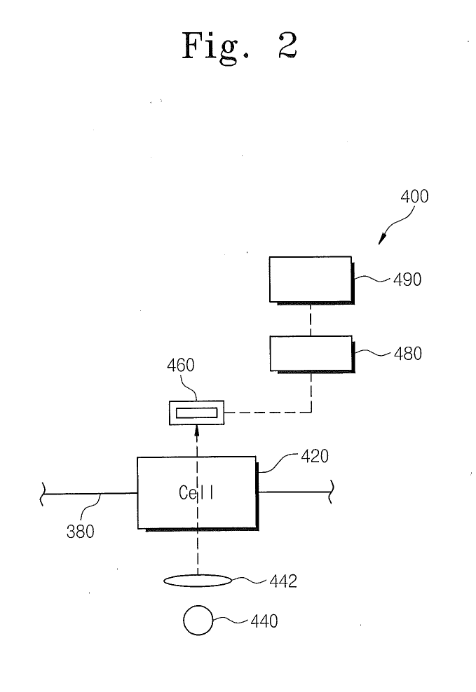

The compound supply pipe 380 may be equipped with a measuring unit 400. The measuring unit 400 may irradiate light on the liquid compound and measure an absorption rate of the liquid compound at a predetermined wavelength. The measuring unit 400 may be used with a spectrometer operable in spectrometry of flow cell type. FIG. 2 is a schematic diagram illustrating a structure of the measuring unit 400 shown in FIG. 1.

Referring to FIG. 2, the measuring unit 400 may include a cell 420, a light source 440, a detector 460, an analogue-to-digital converter (ADC) 480, and a signal processor 490. The cell 420 may be inserted into the compound supply pipe 380, including a path through which the liquid compound flows. Along the path, the light source 440 for irradiating light may be placed at a side of the cell 420. A lens 442 may be interposed between the light source 440 and the cell. The detector 460 may receive light transmitted through the cell 420. The light received by the detector 460 may be converted to a digital or analog signal by the ADC 480, and the converted signal may be transferred to the signal processor 490. The signal processor 490 may find an absorption rate by the liquid compound and detect the concentration of a metal from the liquid compound.

The compound supply pipe 380 may also include a flux gauging member 382 (See FIG. 1) for measuring flux of the liquid compound flowing therethrough. For example, the flux gauging member 382 may be a flowmeter. A pressure sensor as the flux gauging member 382 may be used for indirectly measuring flux of the liquid compound. The flux gauging member 382 may be disposed at the front or read dimension of the measuring unit 400.

The compound supply pipe 380 may be connected to a waste reservoir 390. The liquid compound that has been detected for metal concentration may be stored in the waste reservoir 390 and exhausted to an external environment through a discharge pipe 392. Also, the vent 162 connected to the gas solution reservoir 100 and the vent 164 connected to the bubble eliminator 182 may lead to the waste reservoir 390. Air and bubbles removed from the gas solution reservoir 100 and the bubble eliminator 182 may flow into the waste reservoir 390 and then be exhausted to an external environment through the discharge pipe 392.

Regarding metals, solvents and reagents used in the aforementioned apparatus in accordance with embodiments of the present invention, copper represents an exemplary metal to be detected. The solvent may be selected from solvents that are able to dissolve copper. For example, the solvent may include an acid or deionized water. However, if an acid is used as the solvent, there may be a problem of eroding the solution supply pipe 180 or the compound supply pipe 380. Accordingly, in some embodiments, deionized water is used as the solvent.

The reagent may include a chelating agent for generating a chelate compound by forming coordinate covalent bonds with a metal such as copper. The chelating agent may include aquaion, 4-[2-pyridylazo] resorcinol [(C₅H₄N—N═C₆H₃(OH)₂], bathocuproine [(CH₃)₂(C₆H₅)₂C₁₂H₄N₂], biscyclohexanon oxaldihyrazone [C₆H₁₀C₂H₂N₅O₂C₆H₁₀], diethanolamine [(HOCH₂CH₂)₂NH], or lead diethyldithiocarbamate [Pb(SCSN(C₂H₅OH)₂-C₂H₅OH]. Notably, the reagent may include another type of substance that is capable of being chemically combined with copper while being compounded with the solvent including copper.

If the chelating agent is compounded with the deionized water including metal ions (such as copper), the metal ions to be dissolved in the deionized water are combined with the chelating agent by coordinate covalent bonds, resulting in a chelating compound. Irradiating light on a compound of a chelate and deionized water , without metal (hereinafter, referred to as the “reference compound”), an absorption rate of the reference compound is different from that of the compound of deionized water and the chelating compound combined with metal by coordinate covalent bonds (hereinafter, referred to as the “detection compound”). By comparing the detection compound with the reference compound in absorption rate, the concentration of metal from the detection compound can be determined. Detection can be accomplished by comparing the absorption rate of the detection compound with the absorption rate of the reference compound at a specific wavelength. In particular embodiments, a wavelength associated with a larger difference between the absorption rates therebetween is selected. It is possible to discern the concentration of metal, such as copper, from graphic patterns showing absorption rates of the detection compound along wavelengths. The chelating agent may be of the type that is useful in distinguishing a difference between the absorption rates between the reference and detection compounds at a specific wavelength.

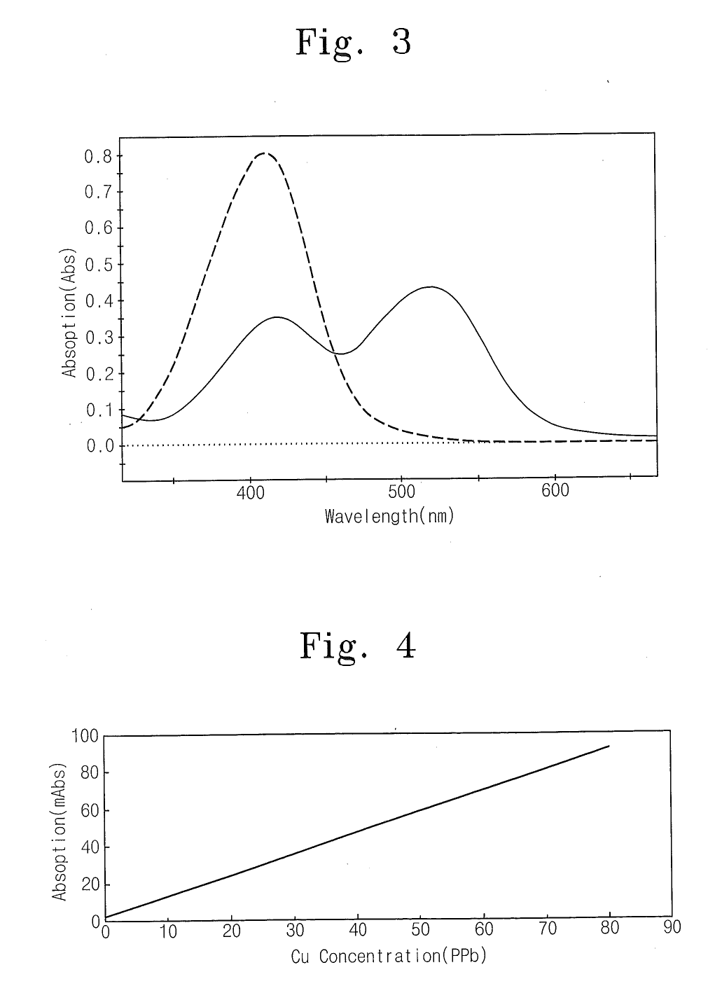

FIG. 3 is a graphic diagram comparatively showing absorption rates of the reference compound and the detection compound with copper when 4-[2-pyridylazo] resorcinol is used as the chelating agent. In FIG. 3, the dotted curve denotes the absorption rates of the reference compound, while the solid curve denotes the absorption rates of the detection compound. Referring to FIG. 3, at a wavelength of about 520 nanometer, the absorption rate of the reference compound is very low, however, the absorption rate of the detection compound is maximized. Therefore, it is desirable to measure the concentration of copper having the absorption rate at about 520 nanometer. However, it is also possible to detect copper concentration at another wavelength. The selection of which may be determined by one skilled in the art.

FIG. 4 is a graphic diagram showing absorption rates of the detection compound versus concentration of copper at a wavelength of about 520 nanometer when 4-[2-pyridylazo] resorcinol is used as the chelating agent. As shown in FIG. 4, it can be seen that according to an elevation of copper concentration, the absorption rate increases linearly. For example, assuming that the copper concentration is X and the absorption rate is Y, the relationship between the copper concentration and the absorption rate can be expressed as Y=1.12X+2.33. According to the graph shown in FIG. 4, the concentration of copper can be readily detected by measuring the absorption rate at a wavelength of about 520 nanometer.

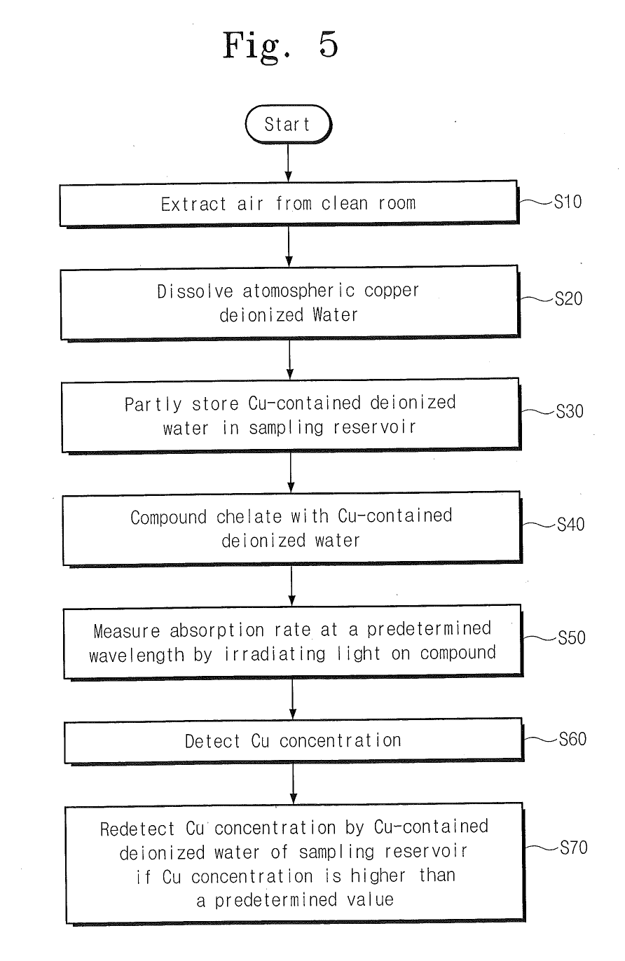

Hereinafter, methods for detecting the concentration of copper by means of the apparatus 20 shown in FIG. 1 will be described. FIG. 5 is a flow chart showing a method for detecting copper concentration according to embodiments of the present invention. Referring to FIG. 5, air may be partly introduced into the apparatus 20 from the clean room 10. The introduced air may be taken from a region around which there is equipment for depositing copper films on wafers (step S10).

The introduced air may flow into the gas solution reservoir 100 that contains the deionized water. During inflow, foreign substances with relatively large size are blocked by the filter. Copper particles of the air are dissolved in the deionized water contained in the gas solution reservoir 100 and the air is discharged from the gas solution reservoir 100 through the vent 162 (step S20). Thereby, the deionized water in which copper is dissolved (i.e., copper-containing deionized water) is at least partly stored in the sampling reservoir 500 (step S30).

Subsequently, the copper-containing deionized water may be compounded with the chelating agent. The copper-containing deionized water and the chelating agent may be compounded by flowing through the first and second mixers 320 and 340. By mixture, the chelating agent is combined with the copper ions, which are dissolved in the deionized water, by coordinate covalent bonding, resulting in a chelating compound (step S40).

The deionized water containing the chelating compound may flow and pass through the path of the cell 420. By irradiating light thereon to transmit the path of the cell 420, an absorption rate may be measured at a specific wavelength (e.g., about 520 nanometer) (step S50). From the measured value of the absorption rate, as shown in FIG. 4, the concentration of copper can be detected (step S60).

During the process of detecting copper concentration, flux may be continuously measured in the compound supply pipe 380. If the flux of the compound supply pipe 380 is out of the range of a reference value, such may indicate that there may be stoppage in the compound supply pipe 380 and an operator may be informed of the abnormal flux by way of a noise or ramp.

If the copper concentration detected by the measuring unit 400 is out of the range of a predetermined value, the operator may redetect the concentration of copper by means of using a solution contained in the sampling reservoir 500. This redetection of the copper concentration may be carried out more precisely by the operator (step S70).

According to embodiments of the present invention, the concentration of a metal (e.g., copper) may be detected from a space such as a clean room that is strictly controlled to guard against pollution.

The above-disclosed subject matter is to be considered illustrative and exemplary, and not restrictive, and the appended claims are intended to cover all such modifications, enhancements, and other embodiments, which fall within the true spirit and scope of the present invention.

What is claimed is:

1. An apparatus for detecting a concentration of a metal in a region, comprising:
a first reservoir in which the metal in the region is dissolved by a solvent;
a second reservoir capable of containing a reagent chemically combined with the metal;
a first unit capable of receiving a solution, in which the metal is dissolved, from the first reservoir and a reagent contained in the second reservoir, and combining the solution and the reagent; and
a second unit capable of irradiating light on a liquid compound provided by the first unit and further capable of detecting the concentration of the metal by measuring an absorption rate of the liquid compound.

2. The apparatus of claim 1, further comprising a reservoir capable of storing at least a portion of the solution in which the metal is dissolved.

3. The apparatus of claim 2, further comprising:
a gas inflow member coupled to an induction member capable of forcing air to be extracted from the region, thereby supplying the air into the first reservoir;
a solution supply member capable of supplying the solution, in which the metal is dissolved, into the first unit from the first reservoir;
a reagent supply member capable of supplying the reagent to the first unit from the second reservoir; and
a compound supply member capable of allowing the liquid compound to flow from the first unit, wherein the second unit is installed on the compound supply member.

4. The apparatus of claim 3 further comprising a reservoir capable of storing at least a portion of the solution supplied through a sampling member from the solution supply member.

5. The apparatus of claim 3 further comprising a flux gauging member capable of measuring flux of the liquid compound in the compound supply member.

6. The apparatus of claim 1 further comprising a vent unit to exhaust internal gas from the first reservoir.

7. The apparatus of claim 3, wherein the reagent and solution supply members each comprise a filter.

8. The apparatus of claim 1, wherein the second reservoir comprises a reservoir having a chelating agent, and the first solution reservoir comprises a reservoir having deionized water or acid.

9. The apparatus of claim 1, wherein the metal is copper, the solvent is deionized water, and the reagent is 4-[2-pyridylazo] resorcinol [(C₅H₄N—N═C₆H₃(OH)₂].

10. The apparatus of claim 9, wherein the second unit functions to detect a concentration of the copper with reference to an absorption rate of the liquid compound at about 520 nanometer.
